(19) United States
(12) Patent Application Publication
(10) Pub. No.: US 2009/0131916 A1
(43) Pub. Date: May 21, 2009

(54) URINATION PANTS

(76) Inventors: Chin-Hung Chiu, Taichung County (TW); Ju-Hui Chiu, Taichung County (TW); Chin-Long Chiu, Taichung County (TW)

Correspondence Address: Chien-Hui Su, P. O. Box 70-121 Taichung, Taichung City, 40899, TW

(21) Appl. No.: 11/943,589

(22) Filed: Nov. 21, 2007

(51) Int. Cl. A61M 27/00

(52) U.S. Cl. 604/544

(57) ABSTRACT

A urination pants comprises a pants body having three openings; an upper opening serving to enclose the wrist of the users; the other two openings serving to receive the legs of the user; a urine guide device being a tube like body and being engaged to the pants body; a hollow receiving unit being connected to the upper opening of the urine guide device; a lower opening of the hollow receiving unit being connected to the upper opening of the urine guide device; and a lower opening of the urine guide device being formed as a urine draining portion. The hollow receiving unit has a tapered shape with a lower opening being connected to the upper opening of the urine guide device. A middle section of the urine guide device is formed with a bending portion.

### FIELD OF THE INVENTION

The present invention relates to urination pants, wherein the woman dressing the urination pants can urinate with a stand pose. The urine will be drain out effectively without pollute the body of the woman.

### BACKGROUND OF THE INVENTION

There are many techniques and devices being employed to enable a female to urinate in a nonsitting position. Some of these methods require a woman to wear an absorbent pad, similar to feminine napkins. Other techniques are invasionary in nature, requiring the insertion of the device into the urethra. Yet other apparatuses involve the use of an intricate device to control the stream of urine for collection or for disposal in a urinal or toilet. They are: Inventor U.S. Pat. No. 2,467,585 Bernard Cohen U.S. Pat. No. 2,522,008 Beatrice Wohlman U.S. Pat. No. 2,522,009 Beatrice Wohlman U.S. Pat. No. 2,852,780 George Gold U.S. Pat. No. 3,224,448 George L. Diebold U.S. Pat. No. 3,398,739 Nicholas A. Marino U.S. Pat. No. 3,864,759 Akira Horiuchi U.S. Pat. No. 4,023,216 Laurie Rhea Li U.S. Pat. No. 4,270,539 Michaud (NASA) U.S. Pat. No. 4,528,703 Richard J. Kraus U.S. Pat. No. 4,608,046 Keivan Towfigh Urinary aid devices that work in conjunction with panty-type and belt-type garments are known in the prior art. The Michaud (NASA) patent discloses a collection device for females which provides a garment for supporting such a device. However, the shape of the urine collection device does not precisely conform to the contours of the genital region of the user, and the rigid structure of the collection device does not entirely prevent leakage. Also, the panty-type garment does not have an exact means of supporting the rigid structure of the collection device.

U.S. patents to Hall and Einhorn disclose urinary devices to be used with belt-type garments. The drainage devices of these patents are not entirely shaped so as to fit the specific contours of the female genital region, and, thus, the belt-type garments do not altogether properly support the drainage devices disclosed.

The above referenced patents disclose urinating devices and devices with garments of various sorts and of various manufacturers, and the like, as well as the methods of their construction, but none of them whether taken singly or in combination, discloses the specific details or combination of the invention in such a way as to bear upon the claims of the present invention.

No prior art has been located which describes the combined features of the urinary-guide apparatus of the invention. None of these prior devices or methods employs the combination of components of the present invention, namely, a noninvasive, one-piece, molded-guide unit made of flexible, semirigid material, which can be cleaned and reused, or of a molded papar or paper-like material intended for disposal after one use, which contours to the female anatomy, and which may be secured and stored in specially constructed garments.

### SUMMARY OF THE INVENTION

Accordingly, the primary object of the present invention is to provide a urination pants, wherein the woman dressing the urination pants can urinate with a stand pose. The urine will be drain out effectively without pollute the body of the woman.

To achieve above objects, the present invention provides a urination pants, comprising: a pants body having three openings; an upper opening serving to enclose the wrist of the users; the other two openings serving to receive the legs of the user; a urine guide device being a tube like body and being engaged to the pants body; a hollow receiving unit being connected to the upper opening of the urine guide device; a lower opening of the hollow receiving unit being connected to the upper opening of the urine guide device; and a lower opening of the urine guide device being formed as a urine draining portion. The hollow receiving unit has a tapered shape with a lower opening being connected to the upper opening of the urine guide device. A middle section of the urine guide device is formed with a bending portion. A middle section of the urine guide device is formed with a bending portion. The urine guide device is engaged to the pants body by high frequency combination or by seeming. An upper opening of the urine guide device being expanded with an annular protecting sheet. The urine draining portion is made of soft material so that it is bendable for storage. The upper edge of the hollow receiving unit is chamfered.

The various objects and advantages of the present invention will be more readily understood from the following detailed description when read in conjunction with the appended drawing.

### BRIEF DESCRIPTION OF THE DRAWINGS

FIG. 1 is a perspective view of the urination pants of the present invention.

FIG. 2 is an enlarged view of the urination pants of the present invention.

FIG. 3 is a schematic cross sectional view of the urination pants of the present invention.

FIG. 4 is a schematic view showing the use of the present invention.

FIG. 5 is a schematic view showing the storage state of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

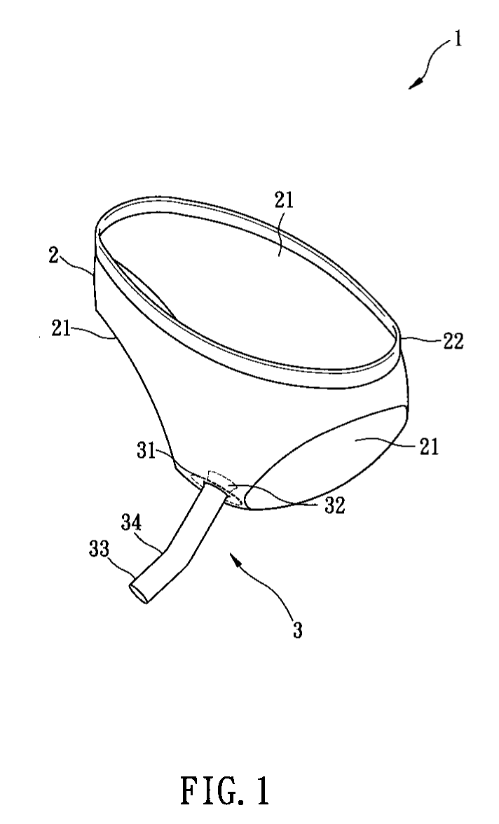

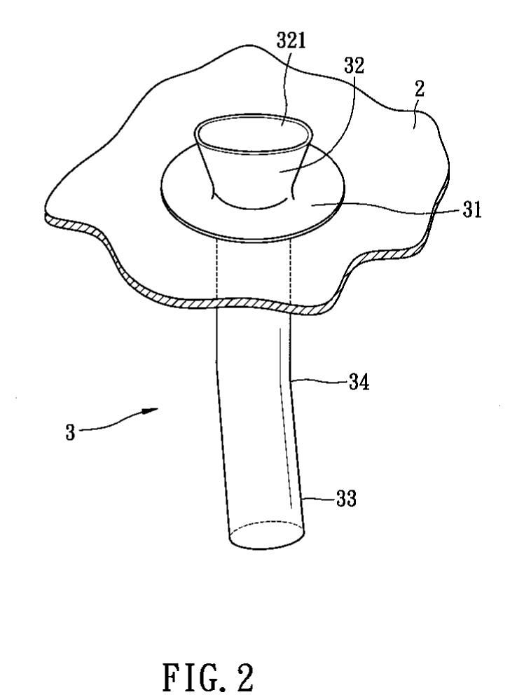

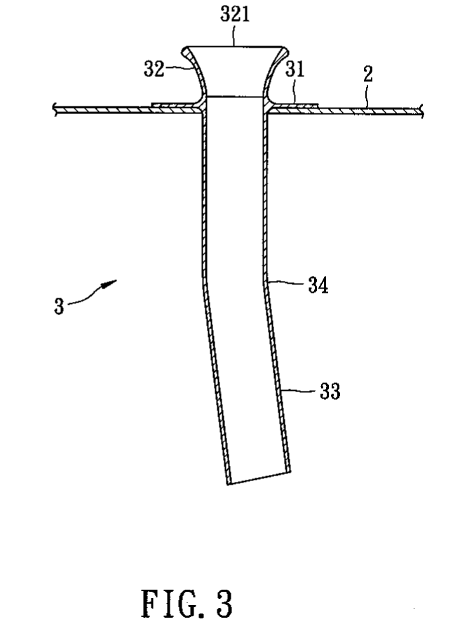

Referring to FIGS. 1 to 3, the urination pants 1 of the present invention is illustrated. The present invention has the following elements.

A pants body 2 has three openings 21. One upper opening 21 serves to enclose the wrist of the users. The other two openings 21 serve to receive the legs of the user. The upper opening 21 is installed with an elastic stripe 22 which provides elasticity to the upper opening 21 of the urination pants 1.

A urine guide device 3 is a tube like body and is engaged to the pants body 2 by high frequency combination or seaming. An upper opening of the urine guide device 3 is expanded with an annular protecting sheet 31. A tapered hollow receiving unit 321 is connected to the upper opening of the urine guide device 3. A lower small opening of the hollow receiving unit 321 is connected to the upper opening of the urine guide device 3. A lower opening of the urine guide device 3 is formed as a urine draining portion 32. A middle section of the urine guide device 3 is formed with a bending portion 34. The upper edge of the hollow receiving unit 321 is chamfered so that the user feels comfortable. The urine draining portion 32 is made of soft material.

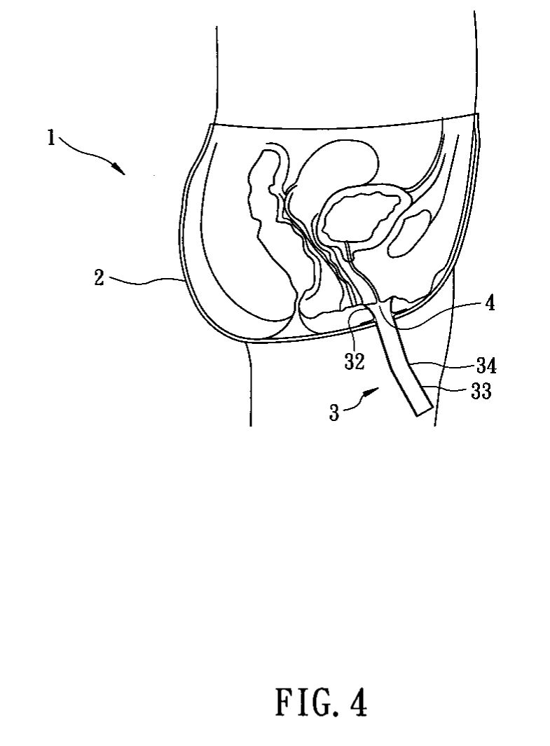

In using, referring to FIG. 4, the protecting sheet 31 resists a woman's urethra so as to retain the stability of the urine guide device 3. Thus, the woman can urinate in standing pose.

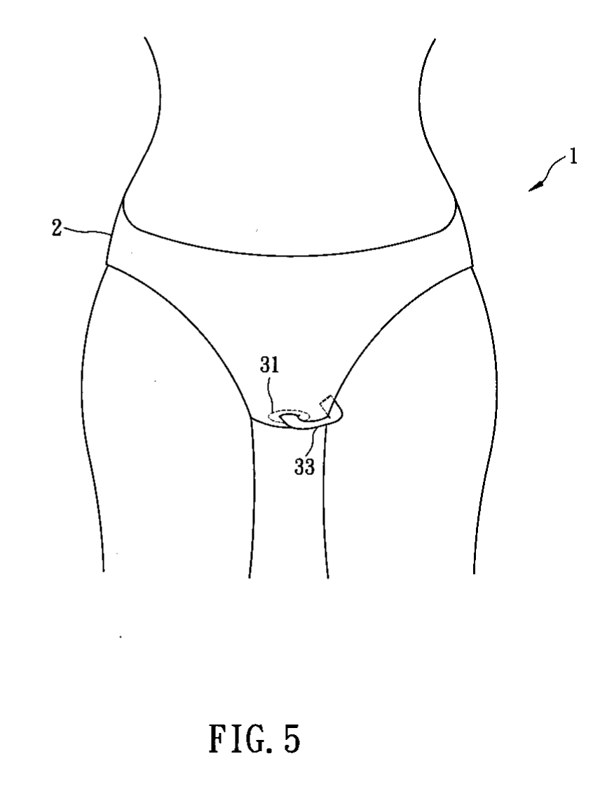

Referring to FIG. 5, it is illustrated that when the present invention is not used, the urine guide device 3 is bent for storage.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A urination pants, comprising:
a pants body having three openings; an upper opening serving to enclose the wrist of the users; the other two openings serving to receive the legs of the user; and
a urine guide device being a tube like body and being engaged to the pants body; a hollow receiving unit being connected to the upper opening of the urine guide device; a lower opening of the hollow receiving unit being connected to the upper opening of the urine guide device; and a lower opening of the urine guide device being formed as a urine draining portion.

2. The urination pants as claimed in claim 1, wherein the hollow receiving unit has a tapered shape with a lower opening being connected to the upper opening of the urine guide device.

3. The urination pants as claimed in claim 1, wherein a middle section of the urine guide device is formed with a bending portion.

4. The urination pants as claimed in claim 2, wherein a middle section of the urine guide device is formed with a bending portion.

5. The urination pants as claimed in claim 1, wherein the urine guide device is engaged to the pants body by high frequency combination or by seeming.

6. The urination pants as claimed in claim 4, wherein an upper opening of the urine guide device being expanded with an annular protecting sheet.

7. The urination pants as claimed in claim 4, wherein the urine draining portion is made of soft material so that it is bendable for storage.

8. The urination pants as claimed in claim 1, wherein the upper edge of the hollow receiving unit is chamfered.
